(19) United States
(12) Patent Application Publication
(10) Pub. No.: US 2025/0355402 A1
(43) Pub. Date: Nov. 20, 2025

(54) SCENTED WATCH

(71) Applicant: Yiwu Baosuo Network Technology Co., Ltd., Jinhua (CN)

(72) Inventor: Anmin WU, Jinhua (CN)

(21) Appl. No.: 19/283,324

(22) Filed: Jul. 29, 2025

(30) Foreign Application Priority Data
Jul. 9, 2025 (CN) 202521436225.1

(51) Int. Cl. G04B 47/00; A44C 5/00; A61L 9/12

(52) U.S. Cl. CPC G04B 47/00; A44C 5/0007; A61L 9/12; A61L 2209/133

(57) ABSTRACT

A scented watch, including a main body, a scenting component and a watch strap. Two ends of the scenting component are hinged to the main body and the watch strap, respectively. The scenting component includes a case, a cover, and a fragrant sheet. The fragrant sheet is placed in an accommodating cavity enclosed by the case and the cover.

### CROSS-REFERENCE TO RELATED APPLICATIONS

This application claims the benefit of priority from Chinese Patent Application No. 202521436225.1, filed on Jul. 9, 2025. The content of the aforementioned application, including any intervening amendments thereto, is incorporated herein by reference in its entirety.

### TECHNICAL FIELD

This application relates to the technical field of watches, and more particularly to a scented watch.

### BACKGROUND

Watches are usually worn on users' wrists as a portable timer to help them manage time in their daily lives. However, with the continuous development of technology, a large number of electronic products with precise timekeeping functions, such as mobile phones and computers, have emerged. The watches are not necessary for the time management any more, and are now generally used as fashionable accessories. Traditional watches are mainly designed for time display, and fail to create a pleasant atmosphere to meet consumers' demands for diversification and personalization.

### SUMMARY

An object of the present disclosure is to provide a scented watch capable of releasing fragrance to create a fragrant atmosphere to address the problem in the prior art.

This application provides a scented watch, comprising:
a main body;
a scenting component; and
a watch strap;
wherein a first end of the scenting component is hinged to the main body; the scenting component comprises a case, a cover and a fragrant sheet; a top surface of the case is provided with an air hole; the cover is arranged on a bottom surface of the case; the case and the cover are configured to enclose to form an accommodating cavity; and the fragrant sheet is arranged in the accommodating cavity; and
the watch strap is hinged to a second end of the scenting component.

In an embodiment, the scented watch further includes a limiting mechanism;
wherein the limiting mechanism comprises a ball plunger and a ball groove;
the ball groove is arranged at the case; and
a first end of the ball plunger is provided on the cover, and a second end of the ball plunger is configured to be engaged with the ball groove to limit the cover and the case.

In an embodiment, the ball plunger includes a ball, a base, and a spring; the ball is partially arranged in the base;
a first end of the spring abuts against the base, and a second end of the spring abuts against the ball; and
an end of the base is arranged at the cover; and the ball is configured to fit the ball groove.

In an embodiment, the bottom surface of the case is provided with a first groove communicated with the air hole;
a top surface of the cover is provided with a second groove; and
the first groove is butt-jointed with the second groove to form the accommodating cavity.

In an embodiment, the cover includes a cover body and a baffle;
the baffle is arranged at an end of the cover body;
a top surface of the cover body is provided with the second groove; and
the cover body and the baffle are respectively configured to cover adjacent two sides of the first groove.

In an embodiment, the bottom surface of the cover body is provided with a push groove.

In an embodiment, a slide groove is provided in the first groove; a side of the cover is provided with a slider; and the slider is slidably connected to the slide groove.

In an embodiment, the number of the slide groove is two, and the number of the slider is two; two slide grooves are respectively arranged on opposite side walls of the first groove; and the two sliders are respectively arranged on opposite side walls of the cover.

In an embodiment, the number of the scenting components is two, and the number of the watch strap is two; the main body is located between two scenting components; and an end of one of two watch straps away from the two scenting components is connected to an end of the other of the two watch straps away from the two scenting components.

In an embodiment, a plurality of air holes are provided, and the plurality of air holes are arranged at intervals at the case.

Compared to the prior art, this application has the following beneficial effects.

Regarding the scented watch provided herein, a scenting component is provided between the main body and the strap, which includes a case, a cover and a fragrant sheet contained in the accommodating cavity enclosed by the case and the cover. The fragrant sheet can sustainedly release the fragrance, and the fragrance can spread to the surrounding environment through the air holes, thereby creating a lasting and comfortable fragrance environment for the user. In addition to the basic timing function, the scented watch can also offer the user a unique fragrance experience through the built-in fragrant sheet, facilitating meeting the user's demand for diversification and personalization.

### BRIEF DESCRIPTION OF THE DRAWINGS

In order to illustrate the technical solutions in the prior art or in the embodiments of the present disclosure more clearly, the accompanying drawings needed in the description of the prior art or the embodiments of the present disclosure will be briefly described below. Obviously, presented in the accompanying drawings are merely some embodiments, instead of all embodiments, of the disclosure, and other drawings can also be obtained by those skilled in the art based on these accompanying drawings without paying creative effort.

FIG. 1 is a schematic diagram of a scented watch according to an embodiment of the present disclosure;

FIG. 2 is a schematic diagram of a scenting component according to an embodiment of the present disclosure;

FIG. 3 is a schematic diagram of the scenting component according to an embodiment of the present disclosure from another perspective;

FIG. 4 is a schematic diagram of a case of the scenting component according to an embodiment of the present disclosure;

FIG. 5 is a schematic diagram of a cover and a fragrant sheet of the scenting component according to an embodiment of the present disclosure; and

FIG. 6 is a schematic diagram of a ball plunger according to an embodiment of the present disclosure.

In the figures: 1—main body; 2—scenting component; 21—case; 211—air hole; 212—first groove; 213—slide groove; 22—cover; 221—cover body; 222—baffle; 223—second groove; 224—slider; 225—push groove; 23—fragrant sheet; 3—watch strap; 4—limiting mechanism; 41—ball plunger; 411—ball; 412—base; and 42—ball groove.

The objects, functional features and advantages of the present disclosure will be described below with reference to the embodiments and drawings.

### DETAILED DESCRIPTION OF EMBODIMENTS

The present disclosure will be further described below with reference to the embodiments and accompanying drawings. Obviously, described below are merely some embodiments of the present disclosure, not all embodiments. Based on the embodiments in the present disclosure, all other embodiments obtained by those of ordinary skill in the art without paying creative labor shall fall within the scope of the present disclosure.

It should be noted that all the directional indications (such as up and down, left and right, front and back, etc.) used herein are only used to explain the relative positional relationship or motion among the components under a specific posture (as shown in the attached figure). If the specific posture changes, the corresponding directional indication will be adjusted accordingly.

Furthermore, the expressions involving “first”, “second”, etc. in the present disclosure are only for descriptive purposes and should not be understood as indicating or implying the relative importance or the quantity of the technical features involved. Therefore, features defined by “first” or “second” can explicitly or implicitly indicate the inclusion of at least one of such features.

In addition, with respect to the term “and/or”, taking “A and/or B” as an example, it includes technical solution A, technical solution B, and a combination thereof. The technical solutions in individual embodiments can be combined with each other as long as the combined solution can be realized by one of ordinary skill in the art. If the combination of technical solutions is contradictory or unachievable, it should be considered infeasible and not fall within the scope of the present disclosure.

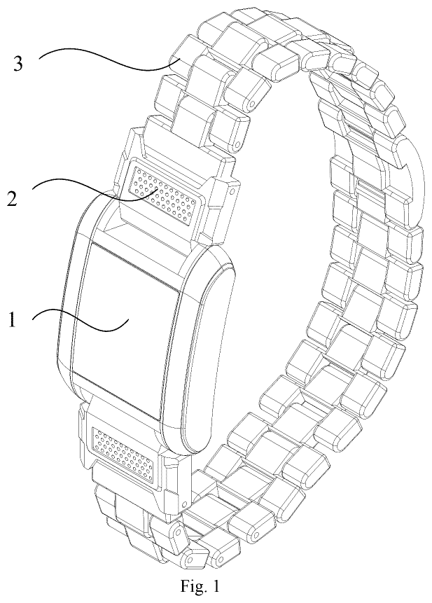

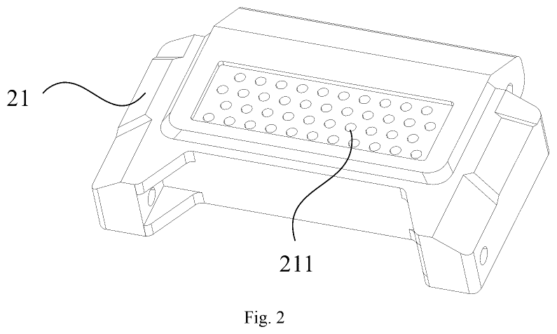

Referring to FIGS. 1-5, the present disclosure provides a scented watch, which includes a main body 1, a scenting component 2 and a watch strap 3. A first end of the scenting component 2 is hinged to the main body 1. The scenting component 2 includes a case 21, a cover 22 and a fragrant sheet 23. The top surface of the case 21 is provided with an air hole 211, and the cover 22 is arranged on a bottom surface of the case 21. The case 21 and the cover 22 are configured to enclose to form an accommodating cavity, and the fragrant sheet 23 is arranged in the accommodating cavity. The watch strap 3 is hinged to a second end of the scenting component 2.

The present disclosure provides a scented watch. By setting a scenting component 2 between the main body 1 and the watch strap 3, the scenting component 2 includes a case 21, a cover 22 and a fragrant sheet 23. The fragrant sheet 23 is arranged in the accommodating cavity and can continuously release a fragrance, which spreads to the surrounding environment through an air hole 211, providing the users with a lasting fragrance experience and creating a fragrant atmosphere to enhance the comfort of the wearer. The scented watch of this application not only can achieve the timekeeping function, but also can provide fragrance for the wearer through the built-in fragrant sheet 23, thereby meeting the wearer's demand for diversification and personalization.

Specifically, one end of the case 21 is hinged to the main body 1, and the other end of the case 21 is hinged to the watch strap 3.

Specifically, a plurality of air holes 211 are provided at intervals on the case 21. The air holes 211 can enable the aroma to be uniformly released, avoiding excessive concentration or uneven diffusion of the aroma.

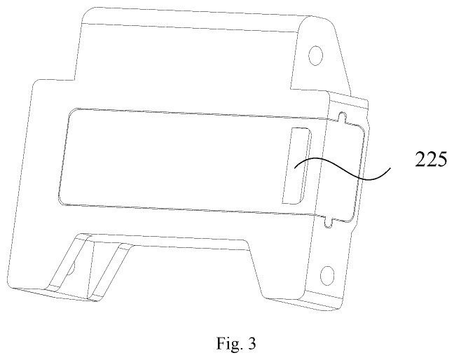

Referring to FIGS. 3-5, the cover 22 includes a cover body 221 and a baffle 222. The baffle 222 is arranged at an end of the cover body 221. A bottom surface of the case 21 is provided with a first groove 212, which is connected to the air hole 211. A top surface of the cover body 221 is provided with a second groove 223. When the case 21, the cover body 221 and the baffle 222 are enclosed, the first groove 212 and the second groove 223 are configured to form an accommodative groove. The cover body 221 blocks the bottom opening of the first groove 212, and the baffle 222 blocks the side opening of the first groove 212.

Specifically, both the first groove 212 and the second groove 223 are rectangular in shape, and the accommodating cavity is also rectangular in shape. The fragrant sheet 23 matches the accommodating cavity, and is rectangular in shape.

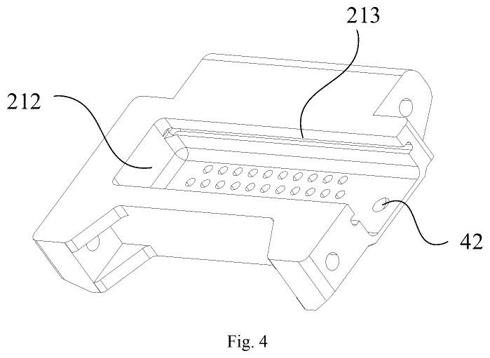

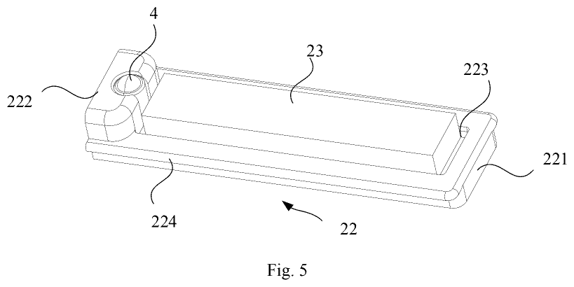

Referring to FIGS. 4-5, a slide groove 213 is provided in the first groove 212, and a slider 224 is provided on the side of the cover body 221. The slider 224 is slidably connected to the slide groove 213.

Specifically, two slide grooves 213 and two sliders 224 are provided. The two slide grooves 213 are respectively arranged on opposite side walls of the first groove 212, and the two sliders 224 are respectively arranged on opposite side walls of the cover body 221.

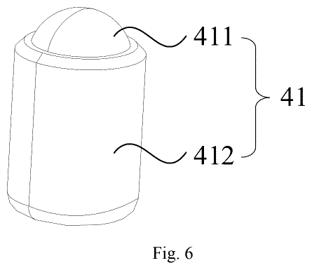

Referring to FIGS. 5-6, the scented watch also includes a limiting mechanism 4. The limiting mechanism 4 includes a ball plunger 41 and a ball groove 42. The case 21 is provided with the ball groove 42. One end of the ball plunger 41 is arranged on the cover 22, and the other end of the ball plunger 41 is configured to be engaged with the ball groove 42 to limit the cover 22 and the case 21.

Specifically, the top surface of the first groove 212 is provided with a ball groove 42, the baffle 222 is provided with an installation hole, and the ball plunger 41 is arranged in the installation hole.

Referring to FIG. 1, the ball plunger 41 includes a ball 411, a base 412 and a spring. The spring is arranged in the base 412, and the ball 411 is partially arranged the base 412. A first end of the spring abuts against the base 412, and a second end of the spring abuts against the ball 411. An end of the base 412 is arranged in the installation hole. The ball 411 of the ball plunger 41 is interlocked with the ball groove 42.

Specifically, the ball 411 is spherical in shape, while the ball groove 42 is hemispherical.

Referring to FIG. 3, the bottom surface of the cover body 221 is also provided with a push groove 225, which facilitates the operator to push the cover 22 to slide relative to the case 21.

When the cover 22 and the case 21 need to be closed, the cover 22 is pushed along the slide groove 213 until the ball 411 is inserted into the ball groove 42, thus achieving a fixed connection between the cover 22 and the case 21. When the cover 22 needs to be separated from the case 21, the cover 22 is pushed to slide along the slide groove 213, and the ball 411 is forced to compress the spring, causing the ball 411 to slide along the ball groove 42 until the ball 411 is separated from the ball groove 42. Thus, the cover 22 can slide out along the slide groove 213, facilitating the quick replacement of the fragrant sheet 23. The limiting mechanism 4 can not only achieve the stable placement of the fragrant sheet 23 to prevent the fragrant sheet 23 from falling during use, but also facilitate the quick removal and replacement of the fragrant sheet 23.

Referring to FIG. 1, two scenting components 2 and two watch straps 3 are provided. The main body 1 is provided between the two scenting components 2, and an end of one of the two watch straps 3 away from the two scenting components 2 is connected to an end of the other of the two watch straps 3 away from the two scenting components 2.

Specifically, the two watch straps 3 are connected through a fastener.

The embodiments disclosed above are merely illustrative of the disclosure, and are not intended to limit the present disclosure. It should be understood that any modifications, changes and replacements made by those skilled in the art without departing from the spirit of the disclosure shall fall within the scope of the disclosure defined by the appended claims.

What is claimed is:

1. A scented watch, comprising:
a main body;
a scenting component; and
a watch strap;
wherein a first end of the scenting component is hinged to the main body; the scenting component comprises a case, a cover and a fragrant sheet; a top surface of the case is provided with an air hole; the cover is arranged on a bottom surface of the case; the case and the cover are configured to enclose to form an accommodating cavity; and the fragrant sheet is arranged in the accommodating cavity; and
the watch strap is hinged to a second end of the scenting component.

2. The scented watch of claim 1, further comprising:
a limiting mechanism;
wherein the limiting mechanism comprises a ball plunger and a ball groove; the ball groove is arranged at the case; a first end of the ball plunger is provided on the cover, and a second end of the ball plunger is configured to be engaged with the ball groove to limit the cover and the case.

3. The scented watch of claim 2, wherein the ball plunger comprises a ball, a base and a spring; the spring is arranged in the base; the ball is partially arranged in the base;
a first end of the spring abuts against the base, and a second end of the spring abuts against the ball; an end of the base is arranged at the cover; and the ball is configured to fit the ball groove.

4. The scented watch of claim 1, wherein the bottom surface of the case is provided with a first groove communicated with the air hole; a top surface of the cover is provided with a second groove; and the first groove is butt-jointed with the second groove to form the accommodating cavity.

5. The scented watch of claim 4, wherein the cover comprises a cover body and a baffle; the baffle is arranged at an end of the cover body; a top surface of the cover body is provided with the second groove; and the cover body and the baffle are respectively configured to cover adjacent two sides of the first groove.

6. The scented watch of claim 5, wherein a bottom surface of the cover body is provided with a push groove.

7. The scented watch of claim 4, wherein a slide groove is provided in the first groove; a side of the cover is provided with a slider; and the slider is slidably connected to the slide groove.

8. The scented watch of claim 7, wherein the number of the slide groove is two, and the number of the slider is two; two slide grooves are respectively arranged on opposite side walls of the first groove; and the two sliders are respectively arranged on opposite side walls of the cover.

9. The scented watch of claim 1, wherein the number of the scenting components is two, and the number of the watch strap is two; the main body is located between two scenting components; and an end of one of two watch straps away from the two scenting components is connected to an end of the other of the two watch straps away from the two scenting components.

10. The scented watch of claim 1, wherein a plurality of air holes are provided, and the plurality of air holes are arranged at intervals at the case.
